SEQUENCE LISTING (51 sequences)

SEQ ID NO: 1 - protein, 1368 aa, Streptococcus pyogenes [sequence omitted]

SEQ ID NO: 2 - protein, 1368 aa, Artificial Sequence
  Dead-SpCas9 (D10A,H840A)
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - DNA, 23 bp, Artificial Sequence
  On Target
actcagaaacaaggacatctggg

SEQ ID NO: 4 - DNA, 23 bp, Artificial Sequence
  Discriminatory target
actcaaaaacaaggacatctggg

SEQ ID NO: 5 - DNA, 20 bp, Artificial Sequence
  20bps guide
actcagaaacaaggacatct

SEQ ID NO: 6 - protein, 1368 aa, Artificial Sequence
  V5
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARANQTTQKGQKNSRERMKRIEEGIKELGSQILKEHPVENTQLQNEKLYLYYLQNGRDMYVDQELDVNRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 1368 aa, Artificial Sequence
  V10
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITTAVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 8 - protein, 1368 aa, Artificial Sequence
  V12
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVEQRQITKRVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 9 - protein, 1368 aa, Artificial Sequence
  V17
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVEGRQITKLVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 10 - protein, 1368 aa, Artificial Sequence
  V19
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVEGRQITKTVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 11 - protein, 1368 aa, Artificial Sequence
  V20
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVEARQITTKVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 12 - protein, 1368 aa, Artificial Sequence
  V1117
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVEYRQITKAVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 13 - protein, 1368 aa, Artificial Sequence
  V1125
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVEGRQITARVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 14 - protein, 1368 aa, Artificial Sequence
  V1137
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVECRGITKAVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 15 - DNA, 4104 bp, Streptococcus pyogenes
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 16 - DNA, 4104 bp, Artificial Sequence
  Dead-SpCas9
atggacaagaagtatagcatcggcctggccatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatgccatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 17 - DNA, 23 bp, Artificial Sequence
  Ggfp_site 12
gcactgcacgccgtaggtcaggg

SEQ ID NO: 18 - DNA, 23 bp, Artificial Sequence
  Gemx1
gagtccgagcagaagaagaaggg

SEQ ID NO: 19 - DNA, 23 bp, Artificial Sequence
  Gemx1 OT1
gagttagagcagaagaagaaagg

SEQ ID NO: 20 - DNA, 23 bp, Artificial Sequence
  Gelane_62
gtcaagccccagaggccacaggg

SEQ ID NO: 21 - DNA, 23 bp, Artificial Sequence
  Gelane_62_OT
gccaaaccccaaaggccacacgg

SEQ ID NO: 22 - protein, 1368 aa, Artificial Sequence; identical to SEQ ID NO: 7
  Variant 1 V10

SEQ ID NO: 23 - protein, 1368 aa, Artificial Sequence
  Variant 2
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKAVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 24 - protein, 1368 aa, Artificial Sequence
  Variant 3
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITTHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 25 - protein, 1368 aa, Artificial Sequence
  Variant 4
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITTYVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 26 - protein, 1368 aa, Artificial Sequence
  Variant 5
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITTDVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 27 - protein, 1368 aa, Artificial Sequence
  Variant 6
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITTTVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 28 - protein, 1368 aa, Artificial Sequence
  Variant 7
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITYAVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 29 - protein, 1368 aa, Artificial Sequence
  Variant 8
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITDAVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - protein, 1368 aa, Artificial Sequence
  Variant 9
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITAAVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 31 - DNA, 4104 bp, Artificial Sequence
  Variant 1 V10
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaacggctgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 32 - DNA, 4104 bp, Artificial Sequence
  Variant 2
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaaggctgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 33 - DNA, 4104 bp, Artificial Sequence
  Variant 3
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaacgcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 34 - DNA, 4104 bp, Artificial Sequence
  Variant 4
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaacgtacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 35 - DNA, 4104 bp, Artificial Sequence
  Variant 5
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaacggacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 36 - DNA, 4104 bp, Artificial Sequence
  Variant 6
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaacgaccgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 37 - DNA, 4104 bp, Artificial Sequence
  Variant 7
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacatacgctgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 38 - DNA, 4104 bp, Artificial Sequence
  Variant 8
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacagacgctgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 39 - DNA, 4104 bp, Artificial Sequence
  Variant 9
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacagccgctgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 40 - DNA, 20 bp, Artificial Sequence
  CXCR4 spacer
atggcaagagacccacacac

SEQ ID NO: 41 - DNA, 421 bp, Artificial Sequence
  CXCR4 On target amplicon
ggaaatgaaacttggggcgaggaccacgggtgcagaccccgttaccttctccacccaggaaaatgccccgctccctaacgtcccaaacgcgccaagtgataaacacgaggatggcaagagacccacacaccggaggagcgcccgcttgggggaggaggtgccgtttgttcattttctgacactcccgcccaatataccccaagcaccgaagggccttcgttttaagaccgcattctctttacccactacaagttgcttgaagcccagaatggtttgtatttaggcaggcgtgggaaaattaagtttttgcgctttaggagaatgagtctttgcaacgcccccgccctccccccgtgatcctcccttctcccctcttccctccctgggcgaaaaacttcttacaaaaagttaatcactgccc

SEQ ID NO: 42 - DNA, 373 bp, Artificial Sequence
  CXCR4 Off target amplicon
gaagcacctgaccagccatgctctgggcctctgccaacctttgccaatgctgggaaccatgcctgccctcccagccttcagcaccaccaccaaggactgaccgcaggcaccctcaggagagccctcggtgggggtgtggcacacggatggcaggagaaccacacacggggctggcggccccaccccagggcacgagttcagctcgggcgcacacgctggcctcacctgggcgagcagtggtgctcctctccctccggcctctcctgtctaggcagcaggccagcctggtgggtgggaagtggcttttcccaagcgaagttgagtgtggacacagtgtggcctccacggcacctcccactgctgccacaggaac

SEQ ID NO: 43 - DNA, 20 bp, Artificial Sequence
  ELANE g35 spacer
agtccgggctgggagcgggt

SEQ ID NO: 44 - DNA, 325 bp, Artificial Sequence
  ELANE g35 On target amplicon
ggcgtctgtttcgtacgtgccctgggtgtccctctgctccccacccgctcccagcccggactgcagcaacaggcaccgtggctagaccctaggagggacttcccaaccctgacaggcggcgggcaggtgggcagggcctcgcagtccagcttccccaccttgtctgcctccacagggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctcc

SEQ ID NO: 45 - DNA, 400 bp, Artificial Sequence
  ELANE g35 Off target amplicon
caccacagggacacactcatcaccatcctggtgcctggtgggcaccccagtgtccaccctgtgccacacatcatggccccctaggcaactgcagcgtttctgcgttttccttggctcagctgccccctcacactccatactccctcggcagcactgttaagagacagtcctggctgggagcaggtggggagaggaggggaggggcagtgagaccagtaacagagaagctaggaacaaatgccagagtccaggcgtgagtgggccatggtgcaggcagaggaggccgagggtgagaggcatttaggagtcataactgactgggcgatggatggatgaatttggaaggaggcctgggtaggcccccacccaaactggaaaacgaggaggaaggtttgcacaa

SEQ ID NO: 46 - DNA, 20 bp, Artificial Sequence
  ELANE alt spacer
gctgcgggaatgggattccc

SEQ ID NO: 47 - DNA, 350 bp, Artificial Sequence
  ELANE alt On target amplicon
gagtgaggaccaagcctgagggcttccgcctggaggaactggcaggggagctggagtcccagctgcgggaaagggattcccaggacccagccccgggctccgggccccgttcctcttcccatctcccccacgatgttccagctgcaacggccgggccaaggctgtcgcaagaaccaacgtcctcctggctccaggacaaggggaggggaagggaaccaacgggcctgtctccaggaataggcggggcgagggcacccggggacacgcagagcctgggccaggctggagtcctgacgtgaccacaggcaagtggacccccatggctctcgggttagggagacaatggccct

SEQ ID NO: 48 - DNA, 401 bp, Artificial Sequence
  ELANE alt Off target amplicon
aattaactgccatcctttggagaacataaaggatatttatgatgaagatttctgtgtcatctttttcttttggaagtcattggggtatccccttttgagcaggttaacttctgaaatgtattttgcttgggatactgcaagttctcattatctcatctaacatcaaaagcatgaaaacagttttcagaagtgtgttagacttaagaatttcaggggtttccagctgagggaaaggaattcccaggcatgtacaacaactcattaagctaaatgaattgctaatgctggggtttgctatggacgcagctttgactgcatctgtcaggctggtacatcttttcacttcactgaagagtgttataggtctagaattatgccacagcaagccagaaagtgggtat

SEQ ID NO: 49 - DNA, 20 bp, Artificial Sequence
  EMX spacer
ttctgtgaatgttagaccca

SEQ ID NO: 50 - DNA, 347 bp, Artificial Sequence
  EMX On target amplicon
gtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggagggggtgggcaaccacaaacccaacatcgatgtcacctccaatgactagggtgggcaaccacaaacccacgagggcagagtgctgcttgctgctg

SEQ ID NO: 51 - DNA, 339 bp, Artificial Sequence
  EMX Off target amplicon
gatgcaagcaagcttttcctgacgccccgcttgtccatgtctaggaaagattaacagagagtctgacaccttttaagatctgacagagaaacatttaccatagactatcacctattttttctgagggctgctacctgtacatctgcacaagattgcctttactccatgcctttcttcttctgctctaactctgacaatctgtcttgccatgccataagcccctattctttctgtaaccccaagatggtataaaagcatcaatgattgggcatttctttgagttttcatattttgtatgactcctgtacatacatgtgcagataataaatttgtatgccc

(19) United States
(12) Patent Application Publication
(10) Pub. No.: US 2022/0154157 A1
(43) Pub. Date: May 19, 2022

(54) NEW ENGINEERED HIGH FIDELITY CAS9

(71) Applicant: EmendoBio Inc., Wilmington, DE (US)

(72) Inventors: David Baram, Tel Aviv (IL); Lior Izhar, Tel Aviv (IL); Asael Herman, Ness-Ziona (IL); Liat Rockah, Rishon LeZion (IL); Nadav Marbach Bar, Rehovot (IL); Rafi Emmanuel, Ramla (IL)

(73) Assignee: EmendoBio Inc., Wilmington, DE (US)

(21) Appl. No.: 17/428,253

(22) PCT Filed: Feb. 4, 2020
(86) PCT No.: PCT/US2020/016551

§ 371 Date: Aug. 3, 2021

Related U.S. Application Data
(60) Provisional application No. 62/801,999, filed on Feb. 6, 2019.

(51) Int. Cl. C12N 9/22; C12N 15/11

(52) U.S. Cl. CPC C12N 9/22; C12N 2310/20; C12N 15/111

(57) ABSTRACT

The present invention is directed to, inter glia, composition and methods for genome editing. Specifically, a non-naturally occurring SpCas9 variant having an amino acid substitution at position K929, H930, or at both position K929 and H930.

This application claims the benefit of U.S. Provisional Application No. 62/801,999 filed Feb. 6, 2019, the contents of which is hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “200204_90841-A-PCT_Sequence_Listing_AWG.txt”, which is 299 kilobytes in size, and which was created on Jan. 30, 2020 in the IBM-PC machine format, having an operating system compatibility with MS-Windows, which is contained in the text file filed Feb. 4, 2020 as part of this application.

### BACKGROUND OF INVENTION

Targeted genome modification is a powerful tool that can be used to reverse the effect of pathogenic genetic variations and therefore has the potential to provide new therapies for human genetic diseases. Current genome engineering tools, including engineered zinc finger nucleases (ZFNs), transcription activator-like effector nucleases (TALENs), and most recently, RNA-guided DNA endonucleases such as CRISPR/Cas, produce sequence-specific DNA breaks in a genome. The modification of the genomic sequence occurs at the next step and is the product of the activity of a cellular DNA repair mechanism triggered in response to the newly formed DNA break. These mechanism may include, for example: (1) classical non-homologous end-joining (NHEJ) in which the two ends of the break are ligated together in a fast but also inaccurate manner (i.e. frequently resulting in mutation of the DNA at the cleavage site in the form of small insertion or deletions) or (2) homology-directed repair (HDR) in which an intact homologous DNA donor is used to replace the DNA surrounding the cleavage site in an accurate manner. In addition, HDR can also mediate the precise insertion of external DNA at the break site. Minimal off-target activity of the initial DNA damage inducer (e.g. Cas9 nuclease) is required for efficient and safe genome editing.

### SUMMARY OF THE INVENTION

Unless otherwise defined, all technical and scientific terms used herein have the same meaning as commonly understood by one of ordinary skill in the art to which the invention pertains. Although methods and materials similar or equivalent to those described herein can be used in the practice or testing of embodiments of the invention, exemplary methods and materials are described below. In case of conflict, the patent specification, including definitions, will control. In addition, the materials, methods, and examples are illustrative only and are not intended to be necessarily limiting.

Disclosed herein are engineered Clustered Regularly Interspaced Short Palindromic Repeats (CRISPRs)/CRISPR-associated protein 9 (Cas9) nucleases with altered and improved target specificity and their use in genomic engineering, epigenomic engineering, genome targeting, genome editing, and in vitro diagnostics.

In some embodiments, there is provided a variant of Streptococcus pyogenes Cas9 (SpCas9) protein with increased specificity as compared to the wild-type protein, as well as methods of using them. Advantageously, when the engineered variant SpCas9 proteins are active in a CRISPR/Cas endonuclease system, the CRISPR/Cas endonuclease system displays reduced off-target editing activity and maintained on-target editing activity relative to a wild-type CRISPR/Cas endonuclease system in which a wild-type SpCas9 is active.

According to some embodiments, there is provided a variant SpCas9 having 80% identity to wild-type SpCas9 and having at least one amino acid substitution at a position selected from: 924, 929, 930, 766 and 830. In some embodiments, the variant SpCas9 comprises an amino acid substitution at position 930. In some embodiments, the variant SpCas9 comprises an amino acid substitution at position 924. In some embodiments, the variant SpCas9 comprises an amino acid substitution at position 929. In some embodiments, the variant SpCas9 comprises an amino acid substitution at one or more position selected from positions: 766 and 830.

According to some embodiments, there is provided a non-naturally occurring SpCas9 variant having an amino acid substitution at position K929, H930, or at both position K929 and H930.

In some embodiments, there is provided a variant of Streptococcus pyogenes Cas9 (SpCas9) protein comprising a sequence that is at least 80% identical to the amino acid sequence of wild-type SpCas9 (SEQ ID NO: 1) and having amino acid substitutions at a position selected from the group consisting of one, some or all of the following positions: T924, K929, and H930. Each possibility represents a separate embodiment of the present disclosure.

In some embodiments, the variant SpCas9 exhibits increased specificity to a target site when complexed with a gRNA targeting the target site compared to a wild-type Cas9 (e.g., SpCas9, listed herein as SEQ ID NO: 1).

According to some embodiments, there is provided a CRISPR/Cas system comprising a variant SpCas9, disclosed herein, complexed with a gRNA for targeting a selected DNA sequence, wherein the CRISPR/Cas system displays at least maintained on-target editing activity of the target DNA sequence and reduced off-target editing activity relative to a wild-type CRISPR/Cas system comprising a wild-type SpCas9 protein.

According to some embodiments, there is provided a method for gene editing having reduced off-target editing activity and/or increased on-target editing activity, comprising:
contacting a target site locus with an active CRISPR/Cas system having a variant Cas9 protein of any one of the variants described herein, wherein the active CRISPR/Cas system displays reduced off-target editing activity and maintained on-target editing activity relative to a wild-type CRISPR/Cas system having a wild-type Cas9 protein.

In some embodiments, the CRISPR/Cas system further comprises a gRNA complexed with the variant Cas9 protein.

In some embodiments, there is provided a variant of Streptococcus pyogenes Cas9 (SpCas9) protein comprising an amino acid sequence of any one of SEQ ID NOs 6-14. In some embodiments, the SpCas9 variant comprises an amino acid sequence selected from any of SEQ ID NOs 22-30.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1: Demonstrates the activity and specificity of V10 compared to WT-SpCas9 by utilizing EMX1 gRNA. Both V10 and WT-SpCas9 demonstrate significant editing activity at the EMX1 target site. For WT-SpCas9 a significant off-target activity is also shown, while V10 does not demonstrate off-target activity.

FIGS. 2A-2C: Demonstrates the activity and specificity of the tested variants (V10 and Variants 3, 4, 5, 6, 7, 8, and 9, with each variants listed as “Mutant” throughout each of the figures) compared to WT-SpCas9 by utilizing CXCR4 gRNA. As shown in FIG. 2A, both WT-Cas9 and the V10 variant show editing activity at the CXCR4 target site, however, notably a significant off-target activity is also demonstrated for WT-SpCas9 while V10 does not demonstrate editing activity at the off-target site. As shown in FIG. 2B and FIG. 2C, all tested variants (Variants 3, 4, 5, 6, 7, 8, and 9) are active at the CXCR4 target site. Notably, as opposed to the significant off-target activity demonstrated for WT-SpCas9, the tested variants demonstrate no editing activity or minimal editing activity at the off-target site.

FIG. 3A and FIG. 3B: Demonstrates the activity and specificity of the tested variants (Variants 3, 4, 5, 6, 7, 8, and 9) compared to WT-SpCas9 by utilizing ELANE g35 gRNA. All tested variants (Variants 3, 4, 5, 6, 7, 8, and 9) are active at the ELANE g35 target site. Notably, as opposed to the significant off-target activity demonstrated for WT-SpCas9, the tested variants demonstrate no editing activity or minimal editing activity at the off-target site.

FIG. 4A and FIG. 4B: Demonstrates the activity and specificity of the tested variants (Variants 3, 4, 5, 6, 7, 8, and 9) compared to WT-SpCas9 by utilizing ELANE g58 alt gRNA. All tested variants (Variants 3, 4, 5, 6, 7, 8, and 9) are active at the ELANE g58 alt target site. Notably, as opposed to the significant off-target activity demonstrated by WT-SpCas9, the tested variants demonstrate no editing activity or minimal editing activity at the off-target site.

### DETAILED DESCRIPTION

The present disclosure provides an engineered Streptococcus pyogenes Cas9 (SpCas9) nuclease exhibiting increased specificity to a target site compared to the wild-type SpCas9. When the engineered SpCas9 nuclease is active in a CRISPR/Cas endonuclease system, the CRISPR/Cas endonuclease system displays reduced off-target editing activity and maintained on-target editing activity relative to a wild-type CRISPR/Cas endonuclease system. In some embodiments, the engineered SpCas9 is an SpCas9 variant. In some embodiments, the engineered SpCas9 comprises amino acid substitutions compared to wild-type SpCas9.

In some embodiments, the SpCas9 variants are at least 80%, e.g., at least 85%, 86%, 87%,88%, 89%, 90%, 91%, 92%, 93%,94%, 95%, 96%, 97%, 98% or 99% identical to the amino acid sequence of SEQ ID NO: 1, e.g., have differences at up to 1%, 2%, 3%, 4%, 5%, 6%, 7%, 8%, 9%, 10%, 11%, 12%, 13%, 14%, 15%, or 20% of the residues of SEQ ID NO:1 replaced, e.g., with conservative mutations, in addition to the mutations described herein, or with mutations in addition to the mutations described herein. In some embodiments, the variant retains a desired activity of the parent, e.g., the nuclease activity (except where the parent is a nickase or a dead Cas9), and/or the ability to interact with a guide RNA and target DNA. In some embodiments, the variant retains the desired activity of the parent at a level greater than or equal to the level of activity of the parent. In some embodiments, the variant retains the desired activity of the parent at a level of at least 100%, 95%, 90%, 80%, 70%, 60%, 50%, 40%, or 30% the level of activity of the parent.

In some embodiments, there is provided a variant of Streptococcus pyogenes Cas9 (SpCas9) protein comprising a sequence that is at least 80% identical to the amino acid sequence of wild-type SpCas9 (SEQ ID NO: 1) and having amino acid substitutions at one, two, or three of the following positions: 924, 929, and 930. Each possibility represents a separate embodiment of the present disclosure. In some embodiments, there is provided a variant of Streptococcus pyogenes Cas9 (SpCas9) protein comprising a sequence that is at least 80% identical to the amino acid sequence of wild-type SpCas9 (SEQ ID NO: 1) and having amino acid substitution at one, two, or three of the following positions: T924, K929, and H930. Each possibility represents a separate embodiment of the present disclosure. In some embodiments, the variant of Streptococcus pyogenes Cas9 (SpCas9) protein comprises at least one additional substitution at position Q926.

In some embodiments, the variant of SpCas9 protein comprises amino acid substitutions at the following positions: T924, K929, and H930. In some embodiments, the variant of SpCas9 protein comprises amino acid substitutions at the following positions: T924 and H930. In some embodiments, the variant of SpCas9 protein comprises amino acid substitutions at the following positions: K929 and H930. In some embodiments, the variant of SpCas9 protein comprises amino acid substitution at position H930. In some embodiments, the variant of SpCas9 protein comprises amino acid substitution at position H930 and a second position selected from positions: K929 and T924. In some embodiments, the variant of SpCas9 protein comprises amino acid substitution at position H930 and optionally one or two additional substitutions selected from: K929 and T924.

In some embodiments, the amino acid substitution in position 930 is selected from: H930A, H930L, H930T, H930K and H930R. In some embodiments, the amino acid substitution in position 924 is selected from: T924Q, T924G, T924A, T924Y, and T924C. In some embodiments, the amino acid substitution in position 929 is selected from: K929T and K929A.

In some embodiments, the variant of SpCas9 protein comprises the following amino acid substitutions: K929T and H930A. In some embodiments, the variant of SpCas9 protein comprises the following amino acid substitutions: T924Q and H930R. In some embodiments, the variant of SpCas9 protein comprises the following amino acid substitutions: T924G and H930L. In some embodiments, the variant of SpCas9 protein comprises the following amino acid substitutions: T924G and H930T. In some embodiments, the variant of SpCas9 protein comprises the following amino acid substitutions: T924A, K929T, and H930K. In some embodiments, the variant of SpCas9 protein comprises the following amino acid substitutions: T924Y and H930A. In some embodiments, the variant of SpCas9 protein comprises the following amino acid substitutions: T924G, K929A, and H930R. In some embodiments, the variant of SpCas9 protein comprises the following amino acid substitutions: T924C, Q926G and H930A.

In some embodiments, there is provided a variant of Streptococcus pyogenes Cas9 (SpCas9) protein comprising a sequence that is at least 80% identical to the amino acid sequence of wild-type SpCas9 (SEQ ID NO: 1) and having amino acid substitutions at one or both of the following positions: 766 and 830. Each possibility represents a separate embodiment. In some embodiments, the variant of SpCas9 protein comprises the following amino acid substitutions: E766A and I830V.

In some embodiments, the SpCas9 variants further comprises one or more of a nuclear localization sequence (NLS), cell penetrating peptide sequence, and/or affinity tag. In an embodiment, the SpCas9 variant comprises one or more nuclear localization sequences of sufficient strength to drive accumulation of a CRISPR complex comprising the CRISPR nuclease in a detectable amount in the nucleus of a eukaryotic cell.

In some embodiments, the SpCas9 variant comprises amino acid substitutions selected from amino acid substitutions corresponding to SEQ ID NOs 6-14 as indicated in Table 3, compared to WT SpCas9. In some embodiments, the SpCas9 variant comprises amino acid substitutions selected from amino acid substitutions corresponding to SEQ ID NOs 7-14 as indicated in Table 3, compared to WT SpCas9. In some embodiments, the SpCas9 variant comprises amino acid substitutions selected from amino acid substitutions corresponding to SEQ ID NOs 7-13 as indicated in Table 3, compared to WT SpCas9. In some embodiments, the SpCas9 variant comprises an amino acid sequence selected from any of SEQ ID NOs 6-14. In some embodiments, the SpCas9 variant comprises an amino acid sequence selected from any of SEQ ID NOs 7-14. In some embodiments, the SpCas9 variant comprises an amino acid sequence selected from any of SEQ ID NOs 7-13. In some embodiments, the SpCas9 variant comprises an amino acid sequence selected from any one of SEQ ID NOs 22-30.

According to some embodiments, there is provided an isolated variant Cas9 protein comprising one or more substitution mutations, wherein the isolated variant Cas9 protein is active in a CRISPR/Cas system, wherein the CRISPR/Cas system displays reduced off-target editing activity and maintained on-target editing activity relative to a wild-type CRISPR/Cas system. In some embodiments, the one or more substitution mutations are at position H930 and optionally at one or two of positions T924 and K929. In some embodiments, the one or more substitution mutations are at position H930 and T924 and optionally at position K929. In some embodiments, the one or more substitution mutations are at positions H930 and K929 and optionally at position T924. In some embodiments, the one or more substitution mutations are at position 766 and optionally at position 830. In some embodiments, the one or more substitution mutations are at position 830 and optionally at position 766. In some embodiments, the one or more substitution mutations are at positions 830 and 766.

According to some embodiments, there is provided an isolated variant SpCas9 protein variant comprising a substitution mutation at K929, H930, or both. In some embodiments, the substitution comprises a mutation to a positive, negative, hydrophilic, hydrophobic, polar, or non-polar amino acid. In some embodiments, the substitution corresponds to the mutations listed in Table 5. In some embodiments, the substitution mutation at K929 is selected from any one of the amino acids in the group consisting of R, H, D, E, S, T, N, Q, C, U, G, P, A, I, L, M, F, W, Y and V. In some embodiments, the substitution mutation at H930 is selected from any one of the amino acids in the group consisting of R, K, D, E, S, T, N, Q, C, U, G, P, A, I, L, M, F, W, Y and V.

According to some embodiments, there is provided an isolated nucleic acid encoding a variant Streptococcus pyogenes (SpCas9) protein comprising an amino acid sequence that has at least 80% sequence identity to the amino acid sequence of SEQ ID NO: 1, and having one or more amino acid mutations. In some embodiments the one or more amino acid mutations are at position H930 and optionally at one or more of the following positions: T924 and K929. In some embodiments the one or more amino acid mutations are at positions H930 and T924 and optionally at K929. In some embodiments the one or more amino acid mutations are at positions H930 and K929 and optionally at T924. In some embodiments the one or more amino acid mutations are at positions 830 and 766. In some embodiments the one or more amino acid mutations are at positions 830 and optionally 766. In some embodiments the one or more amino acid mutations are at positions 766 and optionally 830.

According to some embodiments, the amino acid mutations described herein may be applied to corresponding positions in Cas nucleases other than SpCas9. The numerical positions described herein are based on SEQ ID NO: 1, however the corresponding position in other nucleases may not necessarily have the same numerical positional location in the protein sequence, but rather is located in a similar functional or structural domain, or stretch of amino acids, relative to SpCas9.

According to some embodiments, additional mutations to the variant SpCas9 nucleases described herein may be implemented. Examples include, but are not limited to, mutations which alter the PAM recognition sequence, alter the nuclease activity of the enzyme, and truncations or removal of portions of the nuclease. According to some embodiments, the variant SpCas9 may be encoded by any nucleic acid sequence which produces the desired amino acid sequence of the variant. For example, the nuclei acid sequence may be codon-optimized for a cell, such as a bacterial cell, plant cell, or mammalian cell.

In embodiments of the present invention, a CRISPR nuclease and a targeting molecule form a CRISPR complex that binds to a target DNA sequence to effect cleavage of the target DNA sequence. CRISPR nucleases may form a CRISPR complex comprising a CRISPR nuclease and RNA molecule without a further tracrRNA molecule. Alternatively, CRISPR nucleases may form a CRISPR complex between the CRISPR nuclease, an RNA molecule, and a tracrRNA molecule.

According to some embodiments, there is provided a method of gene editing having reduced off-target editing activity and/or increased on-target editing activity, comprising: contacting a target site locus with an active CRISPR endonuclease system having a variant Cas9 protein complexed with a suitable gRNA, wherein the active CRISPR endonuclease system displays reduced off-target editing activity and maintained on-target editing activity relative to a wild-type CRISPR/Cas system.

According to some embodiments, there is provided a non-naturally occurring SpCas9 variant having an amino acid substitution at position K929, H930, or at both position K929 and H930.

In embodiments of the present invention, the amino acid substitution at position K929 is selected from the group consisting of R, H, D, E, S, T, N, Q, C, U, G, P, A, I, L, M, F, W, Y, and V.

In embodiments of the present invention, the amino acid substitution at position K929 is an uncharged, negative, polar or non-polar amino acid.

In embodiments of the present invention, the amino acid substitution at position K929 is selected from the group consisting of K929T, K929Y, K929D, and K929A.

In embodiments of the present invention, the amino acid substitution at position H930 is selected from the group consisting of R, K, D, E, S, T, N, Q, C, U, G, P, A, I, L, M, F, W, Y, and V.

In embodiments of the present invention, the amino acid substitution at position K930 is an uncharged, negative, polar or non-polar amino acid.

In embodiments of the present invention, the amino acid substitution at position H930 is selected from the group consisting of H930A, H930Y, H930D, and H930T.

In embodiments of the present invention, the SpCas9 variant has an amino acid sequence selected from the group consisting of SEQ ID NOs: 22-30.

In embodiments of the present invention, the SpCas9 variant has at least 80% sequence identity to the wild-type SpCas9 amino acid sequence listed as SEQ ID NO: 1.

In embodiments of the present invention, the SpCas9 variant further comprises a nuclear localization sequence (NLS).

In embodiments of the present invention, the variant exhibits increased specificity toward a DNA target site when complexed with a gRNA that targets the said DNA target site compared to a wild-type SpCas9 complexed with the gRNA.

According to some embodiments, there is provided a CRISPR/Cas system comprising the variant SpCas9 of any one of the embodiments described herein, complexed with a gRNA that targets a DNA target site, wherein the CRISPR/Cas system displays reduced off-target editing activity relative to a wild-type CRISPR/Cas system comprising a wild-type SpCas9 protein and the gRNA.

According to some embodiments, there is provided a method for gene editing having reduced off-target editing activity, comprising contacting a DNA target site with an active CRISPR/Cas system comprising a variant SpCas9 protein of any one of the embodiments described herein, wherein the active CRISPR/Cas system displays reduced off-target editing activity relative to a wild-type CRISPR/Cas system comprising a wild-type SpCas9 protein.

In embodiments of the present invention, the gene editing occurs in a eukaryotic cell.

In embodiments of the present invention, in the cell is a plant cell or mammalian cell.

In embodiments of the present invention, the DNA target site is located within or in proximity to a pathogenic allele of a gene.

In embodiments of the present invention, the DNA target site is located in a gene selected from the group consisting of ELANE, CXCR4, EMX, RyR2, KNCQ1, KCNH2, SCN5a, GBA1, GBA2, Rhodopsin, GUCY2D, IMPDH1, FGA, BEST1, PRPH2, KRT5, KRT14, ApoA1, STAT3, STAT1, ADA2, RPS19, SBDS, GATA2, and RPE65.

In embodiments of the present invention, the DNA target is repaired with an exogenous donor template.

In embodiments of the present invention, the off-target editing activity is reduced by at least 2-fold, 10-fold, 10²-fold, 10³-fold, 10⁴-fold, 10⁵-fold, or 10⁶-fold.

According to some embodiments, there is provided a modified cell obtained by the method of any one of the embodiments described herein.

In embodiments of the present invention, the cell is capable of engraftment.

In embodiments of the present invention, the cell is capable of giving rise to progeny cells after engraftment.

In embodiments of the present invention, the cell is capable of giving rise to progeny cells after an autologous engraftment.

In embodiments of the present invention, the cell is capable of giving rise to progeny cells for at least 12 months or at least 24 months after engraftment.

In embodiments of the present invention, the cell is selected from the group consisting of a hematopoietic stem cell, a progenitor cell, a CD34+ hematopoietic stem cell, a bone marrow cell, and a peripheral mononucleated cell.

According to some embodiments, there is provided a composition comprising a modified cell of any one of the embodiments described herein and a pharmaceutically acceptable carrier. According to some embodiments, there is provided an in vitro or ex vivo method of preparing the composition, comprising mixing the cells with the pharmaceutically acceptable carrier.

In the context of the invention, “maintained on-target editing activity” refers to the ability of a SpCas9 variant to target a DNA target site that is targeted by a gRNA associated with, and thereby programming, the SpCas9 variant. In some embodiments, the SpCas9 variant maintains on-target editing activity of a DNA target at a percent editing level greater than or equal to the percent editing level of a wild-type Cas9 for the DNA target. In some embodiments, the SpCas9 variant maintains on-target editing activity of a DNA target of at least 100%, 95%, 90%, 80%, 70%, 60%, 50%, 40%, or 30% the level of percent editing of a wild-type Cas9 for the DNA target.

The SpCas9 variant compositions described herein may be delivered as a protein, DNA molecules, RNA molecules, Ribonucleoproteins (RNP), nucleic acid vectors, or any combination thereof. In some embodiments, the RNA molecule comprises a chemical modification. Non-limiting examples of suitable chemical modifications include 2′-0-methyl (M), 2′-0-methyl, 3′phosphorothioate (MS) or 2′-0-methyl, 3′thioPACE (MSP), pseudouridine, and 1-methyl pseudo-uridine. Each possibility represents a separate embodiment of the present invention.

The SpCas9 variants and/or polynucleotides encoding same described herein, and optionally additional proteins (e.g., ZFPs, TALENs, transcription factors, restriction enzymes) and/or nucleotide molecules such as guide RNA may be delivered to a target cell by any suitable means. The target cell may be any type of cell e.g., eukaryotic or prokaryotic, in any environment e.g., isolated or not, maintained in culture, in vitro, ex vivo, in vivo or in planta.

Any suitable viral vector system may be used to deliver RNA compositions. Conventional viral and non-viral based gene transfer methods can be used to introduce nucleic acids and/or SpCas9 variant protein in cells (e.g., mammalian cells, plant cells, etc.) and target tissues. Such methods can also be used to administer nucleic acids encoding and/or SpCas9 variant protein to cells in vitro. In certain embodiments, nucleic acids and/or SpCas9 variant protein are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds.) (1995); and Yu et al., Gene Therapy 1:13-26 (1994).

Methods of non-viral delivery of nucleic acids and/or proteins include electroporation, lipofection, microinjection, biolistics, particle gun acceleration, virosomes, liposomes, immunoliposomes, polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus. See, e.g., Chung et al. (2006) Trends Plant Sci. 11(1):1-4. Sonoporation using, e.g., the Sonitron 2000 system (Rich-Mar) can also be used for delivery of nucleic acids. Cationic-lipid mediated delivery of proteins and/or nucleic acids is also contemplated as an in vivo or in vitro delivery method. See Zuris et al. (2015) Nat. Biotechnol. 33(1):73-80. See also Coelho et al. (2013) N. Engl. J. Med. 369, 819-829; Judge et al. (2006) Mol. Ther. 13, 494-505; and Basha et al. (2011) Mol. Ther. 19, 2186-2200.

Additional exemplary nucleic acid delivery systems include those provided by Amaxa.RTM. Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386, 4,946,787; and 4,897,355) and lipofection reagents are sold commercially (e.g., Transfectam.TM., Lipofectin.TM. and Lipofectamine.TM. RNAiMAX). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiamid et al (2009) Nature Biotechnology 27(7) p. 643).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. However, an RNA virus is preferred for delivery of the RNA compositions described herein. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues. A SpCas9 variant or a nucleic acid expressing the variant, as well as any associated nucleic acids, may be delivered by a non-integrating lentivirus. Optionally, RNA delivery with Lentivirus is utilized. Optionally the lentivirus includes mRNA of the nuclease, RNA of the guide. Optionally the lentivirus includes mRNA of the nuclease, RNA of the guide and DNA donor template. Optionally, the lentivirus includes the nuclease protein variant and guide RNA. Optionally, the lentivirus includes the nuclease protein variant, guide RNA and/or DNA donor template for homology directed repair. Optionally the lentivirus includes mRNA of the nuclease variant, DNA-targeting RNA, and the tracrRNA. Optionally the lentivirus includes mRNA of the nuclease variant, DNA-targeting RNA, and the tracrRNA, and DNA donor template. Optionally, the lentivirus includes the nuclease protein varoamt, DNA-targeting RNA, and the tracrRNA. Optionally, the lentivirus includes the nuclease protein variant, DNA-targeting RNA, and the tracrRNA, and DNA donor template for homology directed repair.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g. Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

At least six viral vector approaches are currently available for gene transfer in clinical trials, which utilize approaches that involve complementation of defective vectors by genes inserted into helper cell lines to generate the transducing agent.

Plasn and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/Plasn was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Packaging cells are used to form virus particles that are capable of infecting a host cell. Such cells include 293 cells, which package adenovirus, AAV, and .psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells. Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with an RNA composition, and re-infused back into the subject organism (e.g., patient). Various cell types suitable for ex vivo transfection are well known to those of skill in the art (see, e.g., Freshney et al., Culture of Animal Cells, A Manual of Basic Technique (3^{rd} ed. 1994)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO—S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells, any plant cell (differentiated or undifferentiated) as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with the nucleases (e.g. ZFNs or TALENs) or nuclease systems (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-.gamma. and TNF-alpha are known (as a non-limiting example see, Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+(panB cells), GR-1 (granulocytes), and Tad (differentiated antigen presenting cells) (as a non-limiting example see Inaba et al., J. Exp. Med. 176:1693-1702 (1992)). Stem cells that have been modified may also be used in some embodiments.

Notably, any one of the SpCas9 variant described herein may be suitable for genome editing in post-mitotic cells or any cell which is not actively dividing, e.g., arrested cells. Examples of post-mitotic cells which may be edited using an SpCas9 variant of the present invention include, but are not limited to, myocyte, a cardiomyocyte, a hepatocyte, an osteocyte and a neuron.

Vectors (e.g., retroviruses, liposomes, etc.) containing therapeutic RNA compositions can also be administered directly to an organism for transduction of cells in vivo. Alternatively, naked RNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, for example, U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17^{th} ed., 1989).

In some embodiments of the present invention, a variant SpCas9 nuclease is utilized to affect a DNA break at a target site to induce cellular repair mechanisms, for example, but not limited to, non-homologous end-joining (NHEJ) or homology-directed repair (HDR). Accordingly, the term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length (or any integer value there between or there above), preferably between about 100 and 1,000 nucleotides in length (or any integer there between), more preferably between about 200 and 500 nucleotides in length. A nucleic acid template may be a single stranded nucleic acid, a double stranded nucleic acid. In some embodiments, the nucleic acid template comprises a nucleotide sequence, e.g., of one or more nucleotides, that corresponds to wild type sequence of the target nucleic acid, e.g., of the target position. In some embodiments, the nucleic acid template comprises a ribonucleotide sequence, e.g., of one or more ribonucleotides, that corresponds to wild type sequence of the target nucleic acid, e.g., of the target position. In some embodiments, the nucleic acid template comprises modified ribonucleotides.

Insertion of an exogenous sequence (also called a “donor sequence,” donor template” or “donor”), for example, for correction of a mutant gene or for increased expression of a wild-type gene can also be carried out. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. See, e.g., U.S. Patent Publication Nos. 20100047805; 20110281361; and 20110207221. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Accordingly, embodiments of the present invention using a donor template for HDR may be DNA or RNA, single-stranded and/or double-stranded and can be introduced into a cell in linear or circular form. In embodiments of the present invention using: (1) a variant nuclease associated with an RNA molecule comprising a guide sequence to affect a double strand break in a gene prior to HDR and (2) a donor template for HDR.

A donor sequence may also be an oligonucleotide and be used for gene correction or targeted alteration of an endogenous sequence. The oligonucleotide may be introduced to the cell on a vector, may be electroporated into the cell, or may be introduced via other methods known in the art. The oligonucleotide can be used to ‘correct’ a mutated sequence in an endogenous gene (e.g., the sickle mutation in beta globin), or may be used to insert sequences with a desired purpose into an endogenous locus.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted. However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus, for example a CCR5 gene, a CXCR4 gene, a PPP1R12c (also known as AAVS1) gene, an albumin gene or a Rosa gene. See, e.g., U.S. Pat. Nos. 7,951,925 and 8,110,379; U.S. Publication Nos. 20080159996; 201000218264; 20100291048; 20120017290; 20110265198; 20130137104; 20130122591; 20130177983 and 20130177960 and U.S. Provisional Application No. 61/823,689).

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In certain embodiments, the donor molecule comprises a sequence selected from the group consisting of a gene encoding a protein (e.g., a coding sequence encoding a protein that is lacking in the cell or in the individual or an alternate version of a gene encoding a protein), a regulatory sequence and/or a sequence that encodes a structural nucleic acid such as a microRNA or siRNA.

This invention provides a modified cell or cells obtained by use of any of the variants or methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cell (HSC), or any cell suitable for an allogenic cell transplant or autologous cell transplant. The variants and methods described herein may also be utilized to generate chimeric antigen receptor T (CAR-T) cells.

This invention also provides a composition comprising these modified cells and a pharmaceutically acceptable carrier. Also provided is an in vitro or ex vivo method of preparing this, comprising mixing the cells with the pharmaceutically acceptable carrier.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease with the targeting sequence serving as the targeting portion of the CRISPR complex. When the molecule having the targeting sequence is present contemporaneously with the CRISPR molecule the RNA molecule is capable of targeting the CRISPR nuclease to the specific target sequence. Each possibility represents a separate embodiment. An RNA molecule can be custom designed to target any desired sequence.

The term “targets” as used herein, refers to a targeting sequence or targeting molecule's preferential hybridization to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

As used herein the term “wild type” is a term of the art understood by skilled persons and means the typical form of an organism, strain, gene or characteristic as it occurs in nature as distinguished from mutant or variant forms. Accordingly, as used herein, where a sequence of amino acids or nucleotides refers to a wild type sequence, a variant refers to variant of that sequence, e.g., comprising substitutions, deletions, insertions. In embodiments of the present invention, an engineered CRISPR nuclease is a variant CRISPR nuclease comprising at least one amino acid modification (e.g., substitution, deletion and/or insertion) compared to the wild-type SpCas9 nuclease of SEQ ID NO:1.

The terms “non-naturally occurring” or “engineered” are used interchangeably and indicate human manipulation. The terms, when referring to nucleic acid molecules or polypeptides may mean that the nucleic acid molecule or the polypeptide is at least substantially free from at least one other component with which they are naturally associated in nature and as found in nature.

The terms “mutant” or “variant” are used interchangeably and indicate a molecule that is non-naturally occurring or engineered.

As used herein the term “amino acid” includes natural and/or unnatural or synthetic amino acids, including glycine and both the D- or L-, optical isomers, and amino acid analogs and peptidomimetics.

As used herein, “genomic DNA” refers to linear and/or chromosomal DNA and/or to plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of DNA sequences at the target site(s) in a genome.

“Eukaryotic” cells include, but are not limited to, fungal cells (such as yeast), plant cells, animal cells, mammalian cells and human cells.

As used herein, the term “modified cells” refers to cells in which a double strand break is affected by a complex of an RNA molecule and the CRISPR nuclease variant as a result of hybridization with the target sequence, i.e. on-target hybridization. The term “modified cells” may further encompass cells in which a repair or correction of a mutation was affected following the double strand break induced by the variant. The modified cell may be any type of cell e.g., eukaryotic or prokaryotic, in any environment e.g., isolated or not, maintained in culture, in vitro, ex vivo, in vivo or in planta.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity.

The terms “protospacer adjacent motif” or “PAM” as used herein refers to a nucleotide sequence of a target DNA located in proximity to the targeted DNA sequence and recognized by the CRISPR nuclease. The PAM sequence may differ depending on the nuclease identity. For example, wild-type SpCas9 recognizes a “NGG” PAM sequence. A skilled artisan will appreciate that embodiments of the present invention disclose RNA molecules capable of complexing with a nuclease, e.g. a CRISPR nuclease, such as to associate with a target genomic DNA sequence of interest next to a protospacer adjacent motif (PAM). The nuclease then mediates cleavage of target DNA to create a double-stranded break within the protospacer.

As used herein, a sequence or molecule has an X % “sequence identity” to another sequence or molecule if X % of bases or amino acids between the sequences of molecules are the same and in the same relative position. For example, a first nucleotide sequence having at least a 95% sequence identity with a second nucleotide sequence will have at least 95% of bases, in the same relative position, identical with the other sequence.

The terms “nuclear localization sequence” and “NLS” are used interchangeably to indicate an amino acid sequence/peptide that directs the transport of a protein with which it is associated from the cytoplasm of a cell across the nuclear envelope barrier. The term “NLS” is intended to encompass not only the nuclear localization sequence of a particular peptide, but also derivatives thereof that are capable of directing translocation of a cytoplasmic polypeptide across the nuclear envelope barrier. NLSs are capable of directing nuclear translocation of a polypeptide when attached to the N-terminus, the C-terminus, or both the N- and C-termini of the polypeptide. In addition, a polypeptide having an NLS coupled by its N- or C-terminus to amino acid side chains located randomly along the amino acid sequence of the polypeptide will be translocated. Typically, an NLS consists of one or more short sequences of positively charged lysines or arginines exposed on the protein surface, but other types of NLS are known.

The term “CRISPR/Cas system” refers to a CRISPR endonuclease system that includes a Cas9 protein, such as the mutants or variants described herein, and a suitable gRNA for targeting a given target DNA sequence. The term “wild-type CRISPR endonuclease system” refers to a CRISPR endonuclease system that includes wild-type Cas9 protein and a suitable gRNA for targeting a given target DNA sequence.

### EXPERIMENTAL EXAMPLES

### Example 1

### Variants Selection

In order to select SpCas9 variants with increased specificity to the target site (increased ratio between On-target cuts and Off-target cuts), substitutions were introduced into the open reading frame of the wild-type SpCas9 sequence (SEQ ID NO: 1). Semi-rational design of library 15 was performed based on combination positions within the helix of the SpCas9 interacting with the minor groove of the RNA-DNA and was obtained using oligonucleotides comprising degenerative codons (NNK) for positions T924, K929 and H930. Error-prone PCR was used to generate the Ep3 library of random mutations between positions 685 and 1026 containing an average of 1 (±3) base substitutions per 1 Kb. To this end, the Cas9 open reading frame of the library pool was cloned into mammalian expression plasmid harboring lentiviral backbone to enable the packaging of the library into lentiviral particles. This plasmid encodes human codon optimized versions of Cas9 harboring the substitutions in amino acids as listed in Table 3 and Table 4 below and is expressed as a polycistronic mRNA with P2A-mCherry, for expression efficiency control.

### Mammalian Screen System for Active Variants:

The plasmids harboring the mutations within an SpCas9 open reading frame were co-transfected into HEK293TN cells with lentiviral packaging plasmids, pGag/Pol, pRev, Pvsv-G using Liopfectamine 3000 reagent (ThermoFisher). The supernatant of the cells was collected 24 and 52 hours post transfection. The viral particles were then concentrated using PEG-it TM Virus Precipitation Solution (5×) (SBI, system biosciences) according to the manufacturer instructions.

To determine the titer of the library, HEK293 cells were transduced with different dilutions of the viroids. 72 hours post transduction, the cells were analyzed by FACS for mCherry signal, which is an indicator for expressed Cas9 molecules. The titer was calculated at a transduction range of 1-20%, to be sure that each cell contains only a single particle, according to the following formula: Titer=(F×C/V)×D.

F=% of transduced cells (mCherry positive).
C=Cell number at the day of transduction.
V=volume of inoculum in ml.
D=Lentivirus dilution factor.

To screen for highly specific evolved SpCas9 variants, we prepared a HEK293 cells system stably transfected with a plasmid expressing EBFP, EGFP and the gRNA of interest (see SEQ ID NO: 4, Table 1), under the regulation of CMV, EF1, and U6 promoters, respectively. The target sequences for the gRNA (on- and off-targets, see SEQ ID NO: 2 and 3, Table 1) were cloned upstream to the fluorescent proteins in a fashion that editing would cause either a gain or a loss of a signal. These cells were transduced with 0.3-0.5 multiplicity of infection (MOI) of the lentivirus library. Seven days following transduction, mCherry, EBFP and EGFP positive cells were sorted. The number of sorted cells was up to 10 times more than the library variation.

Following sorting, genomic DNA was extracted from the cells and used for amplifying the Cas9 sequences, which then were cloned into a shuttle vector that enables the expression of the Cas9 in both bacterial and mammalian cells. The cloned sequences underwent a negative and a positive selection rounds in bacteria as described below.

### Bacterial-Based Negative Selection System:

The sorted and clonal pool of Library 15, plasmids harboring the mutations within SpCas9 open reading frame, were transformed into competent Escherichia coli strain BW25141 (λDE3) containing negative selection plasmid: a low-copy number plasmid of the negative selection with a Kanamycin resistance gene and embedded Discriminatory target site (for FGA rs2070018 Discriminatory target, see Table 1). Following 3 hours of recovery in TB media with 0.1 Mm IPTG (inducer for the Cas9 variant), transformants were plated on selective TB plates containing Carbomycin and 50 ug/ml Kanamycin. The plates were incubated over night at 37C and the next morning colonies were scraped off the plates for a round of positive selection as described below.

### Bacterial-Based Positive Selection System:

For positive selection, the SpCas9 variants that survived the negative selection were transformed into competent Escherichia coli strain BW25141 (λDE3) containing a positive selection plasmid. This positive selection plasmid is a high-copy plasmid, with embedded on-target site (for FGA rs2070018 [‘on target’, see Table 1). This positive selection plasmid also expresses a toxic gene CcdB under control of BAD promoter. Thus, only active SpCa9 variants that cleave the positive plasmid can survive in the presence of arabinose.

Following a 60 min recovery in TB media, transformations were plated on selective TB plates containing Carbomycin and 15 mM arabinose. The plates were incubated over night at 37C and the next morning single colonies were randomly picked.

TABLE 1
Target cleavage sites

| | | |
|---|---|---|
| FGA | On target | ACTCAGAAACAAGGACATCT |
| rs2070018 | | GGG (SEQ ID NO: 2) |
| | Discriminatory | ACTCAAAAACAAGGACATCT |
| | target | GGG (SEQ ID NO: 3) |
| | 20 bps guide | ACTCAGAAACAAGGACATCT |
| | | (SEQ ID NO: 4) |

### Example 2

To test the activity and specificity of the variants, we developed a Reporter System that utilized HEK293 cells system that would enable the detection of editing at on- and off-target sites (see SEQ ID NO: 2 and 3, Table 1) as a gain of signal of EBFP and EGFP. 500 ng plasmids from colonies retrieved from the positive selection in bacteria (described above) were extracted and transfected into the HEK293 system using TurboFect reagent (Thermo Scientific). As controls, cells were transfected with WT-Cas9 and Dead-Cas9. 12 hours following transfection fresh medium was added and 72 h following transfection cells were harvested and the signal of EBFP and EGFP was monitored by FACS. Activity and specificity of the variants was compared to WT-Cas9. Positive EBFP signal and a weak or no EGFP signal indicates for an active and a specific variant.

Active and specific variants obtained were further analyzed for their on-target activity on endogenous ELANE and EMX1 using Indel Detection by Amplicon Analysis (IDAA). Briefly, HeLa cells were seeded into 96 well-plate (3K/well). 24 h later, cells were co-transfected with 65 ng of Cas9 variants plasmid and 20 ng of gRNA plasmid targeting either ELANE or EMX1, using Turbofect reagent (Thermo Scientific). Wild-type (WT) SpCas9 was used as control. 12 hours later, fresh media was added, and 72 hours post transfection, genomic DNA was extracted, and the expected region targeted by the Cas9 was amplified and the product size was analyzed by capillary electrophoreses with a DNA ladder. The intensity of the bands was analyzed using the Peak Scanner software v1.0. The percent of editing was calculated according the following formula:

100%−(Intensity_{not edited band}/Intensity_{total bands})×100

The fidelity (off-target rate) of active variants (≥60% of WT-cas9 activity) was further evaluated by NGS (next generation sequencing) analysis. Briefly, predicted off-target sites for the gRNAs targeting, ELANE and EMX1 were amplified from the same gDNA extracted for the IDAA analysis (See table 2 for the predicted off-target sites in the genome for each gRNA). The indel frequency in each site was calculated using Cas-Analyzer software (www.rgenome.net/cas-analyzer/#!).

TABLE 2
Summary of gRNA and genomic targets

| gRNA | guide RNA Sequence | genomic location (Hg 19) | genomic sequence |
|---|---|---|---|
| Ggfp_site 12 | GCACTGCACGCCGTAGGTC | NA | NA |
| | AGGG(SEQ ID NO: 17) | | |
| Gemx1 | GAGTCCGAGCAGAAGAAGA | chr2:73160982-73161004 | GAGTCCGAGCAGAAGAAGA |
| | AGGG(SEQ ID NO: 18) | | AGGG(SEQ ID NO: 18) |
| Gemx1_OT1 | GAGTCCGAGCAGAAGAAGA | chr5:45359061-45359083 | GAGTTAGAGCAGAAGAAGA |
| | AGGG(SEQ ID NO: 18) | | AAGG(SEQ ID NO: 19) |
| Gelane_62 | GTCAAGCCCCAGAGGCCAC | chr19:859199-859221 | GTCAAGCCCCAGAGGCCAC |
| | AGGG(SEQ ID NO: 20) | | AGGG(SEQ ID NO: 20) |
| Gelane_62_OT | GCCAAACCCCAAAGGCCAC | chr2:230367804-230367826 | GCCAAACCCCAAAGGCCAC |
| | ACGG(SEQ ID NO: 21) | | ACGG(SEQ ID NO: 21) |

### Results:

As demonstrated in Table 3 and Table 4, the tested variants exhibited increased specificity compared to WT SpCas9.

TABLE 3
% editing of on and off-target sites by SpCas9 variants, WT, and Dead SpCas9

| | | | | | | | | | % Activity and | | |
|---|---|---|---|---|---|---|---|---|---|---|---|
| | | | | | | | | | specificity | | |
| | | | | | | | | | as assayed in | | |
| | | | | | | | | | Reporter System | | |
| SEQ | | Substitutions | Amino Acid at | | | | | | | | On/Off |
| ID | Variant | relative to WT | Position No. | | | | | | On | Off | Target |
| NO. | name | SpCas9 | 766 | 830 | 924 | 926 | 929 | 930 | Target | Target | Ratio |
| 1 | WT-Cas9 | | E | I | T | Q | K | H | 12 | 11 | 1 |
| 5 | Dead-Cas9 | | | | | | | | 0.1 | 0.1 | |
| 6 | V5 | E766A; I830V | A | V | | | | | 16 | 2 | 8 |
| 7 | V10 | K929T; H930A | | | T | Q | T | A | 15 | 3 | 5 |
| 8 | V12 | T924Q; H930R | | | Q | Q | K | R | 16 | 2 | 8 |
| 9 | V17 | T924G; H930L | | | G | Q | K | L | 15 | 1 | 15 |
| 10 | V19 | T924G; H930T | | | G | Q | K | T | 14 | 0.1 | 140 |
| 11 | V20 | T924A; K929T; | | | A | Q | T | K | 16 | 1 | 16 |
| | | H930K | | | | | | | | | |
| 12 | V1117 | T924Y; H930A | | | Y | Q | K | A | 21 | 2 | 11 |
| 13 | V1125 | T924G; K529A; | | | G | Q | A | R | 10 | 0.1 | 100 |
| | | H930R | | | | | | | | | |
| 14 | V1137 | T924C; Q926G; | | | C | G | K | A | 22 | 1 | 22 |
| | | H930A | | | | | | | | | |

Each SEQ ID NO. indicated in the first Column of Table 3 represents an amino acid sequence as set forth for naturally occurring Cas9 from S. pyogenes (WT SpCas9, (e.g., comprising amino acid sequence as set forth in SEQ ID NO: 1) with amino acid substitutions as indicated in the 3rd column of the same row.

TABLE 4
Activity and specificity of variants on endogenous sites.

| | % of editing at On- and Off-target sites | | | |
|---|---|---|---|---|
| | | | gELANE_ | gELANE_ |
| Variant | gEMX1_On | gEMX1_OT | 62_On | 62_OT |
| name | (On-target) | (Off-target) | (On-target) | (Off-target) |
| WT- | 52 | 14 | 62 | 35 |
| Cas9 | | | | |
| Dead- | 4 | 0 | 0.4 | 0.1 |
| Cas9 | | | | |
| V5 | 42 | 0 | 72 | 4 |
| V10 | 34 | 0 | 72 | 1 |
| V12 | 26 | 2 | 44 | 11 |
| V17 | 19 | 1 | 56 | 0 |
| V19 | 16 | 0 | 51 | 0 |
| V20 | 22 | 2 | 49 | 0 |
| V1117 | 22 | 1 | 66 | 0 |
| V1125 | 13 | 0 | 52 | 0 |
| V1137 | 41 | 0 | 46 | 1 |

### Example 3

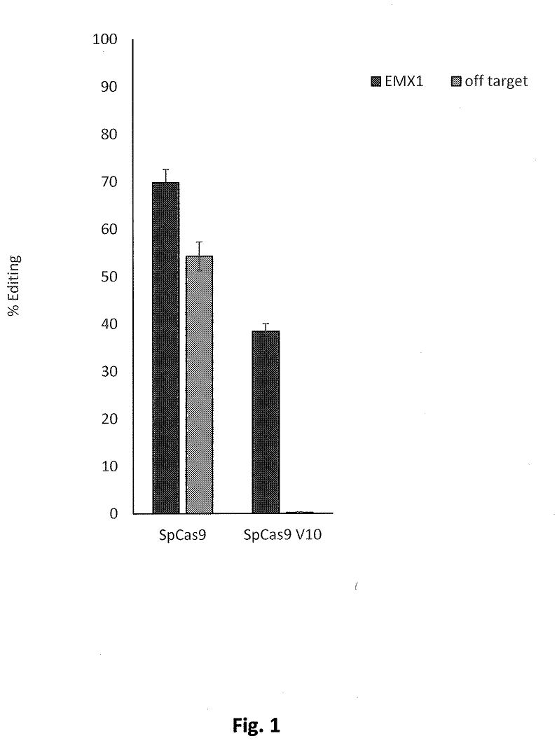

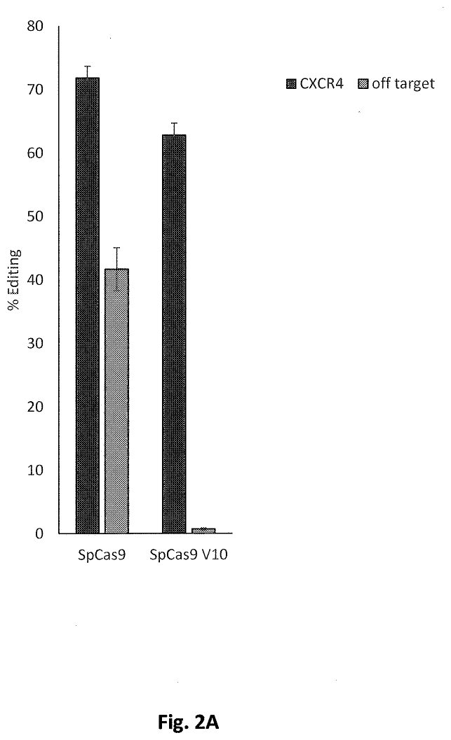

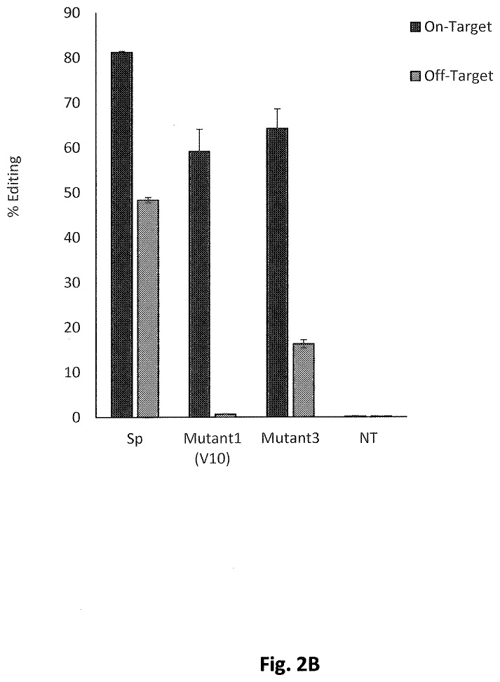

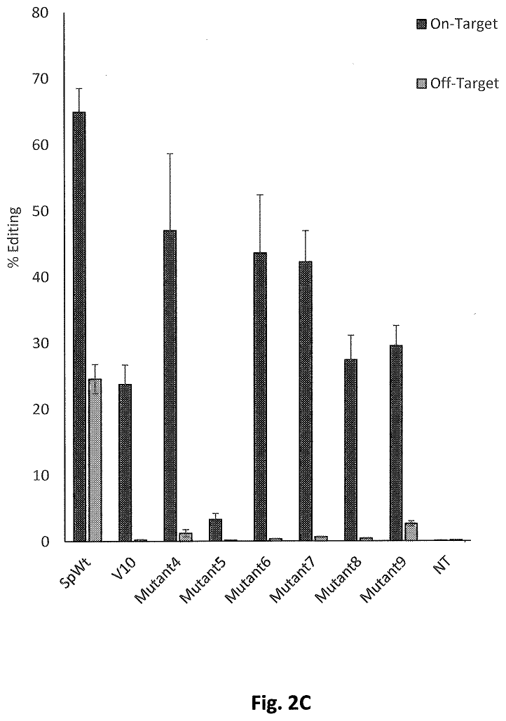

Variant V10 is a combination of two mutations in adjacent positions: K929T and H930A. First its improved specificity was assessed as demonstrated in FIG. 1 and FIG. 2A.

To test the functional role of each of the two mutations comprising variant V10, we constructed a series of mutations representing different amino acid families in position 929 in the context of alanine at position 930, and a second series of mutations representing different amino acid families in position 930 in the context of threonine at position 929 (Table 5). Positive amino acids are represented by lysine or histidine. Negative amino acids are represented by aspartic acid. Polar amino acids are represented by threonine. Hydrophobic amino acids are represented by alanine or tyrosine. These SpCas9 nuclease variants were cloned into pmOMNI plasmid and the nuclease composition was verified by sequencing.

To test the activity and specificity of the variants, we utilized a HeLa cells system that would enable the detection of editing at on- and pre-verified off-target sites (Table 6). HeLa cells were seeded into a 96 well-plate (15K/well). 24 h later, cells were co-transfected with 65 ng of a Cas9 variant plasmid and 20 ng of gRNA plasmid targeting either ELANE g35, ELANE g58_alt or CXCR4 using Jet Optimus reagent (Polyplus transfection). All tests were done in triplicates. As controls, cells were transfected with WT-SpCas9. 6 hours following transfection fresh medium was added and 72 h following transfection cells were harvested, the genomic DNA was extracted, and the expected region targeted by the Cas9 was amplified. Both on-target and pre-validated off target regions were amplified. The level of editing was then determined by indel count extracted from next-generation sequencing (NGS) analysis. Activity and specificity of the variants was compared to WT-SpCas9 and to untreated cells (NT) as a negative control.

### Results:

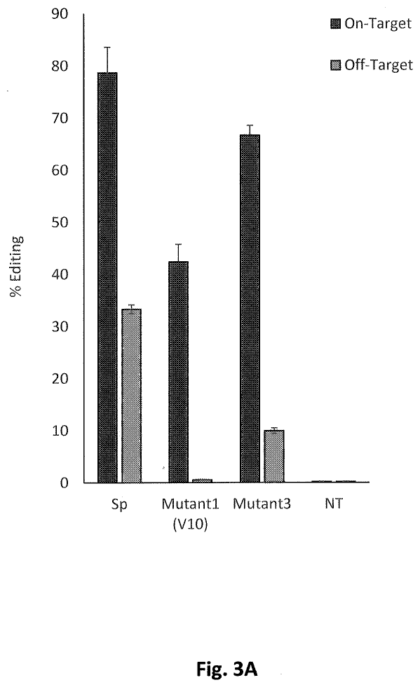

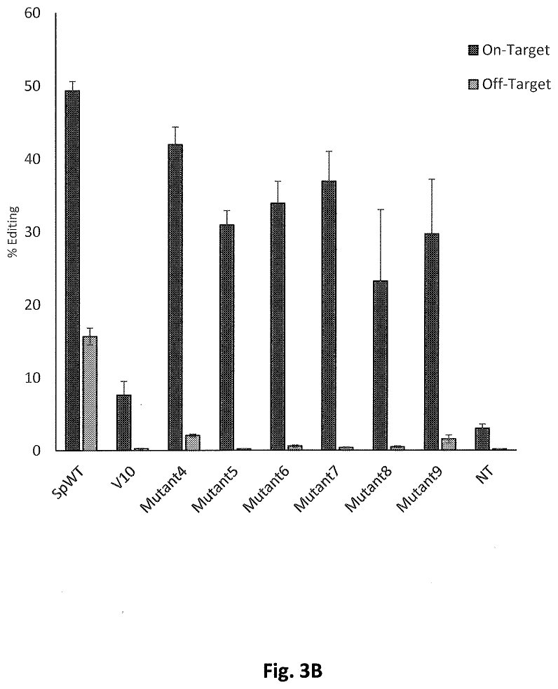

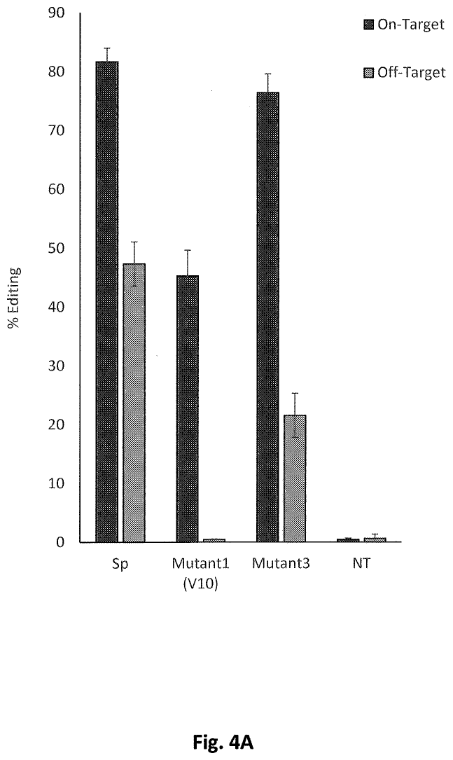

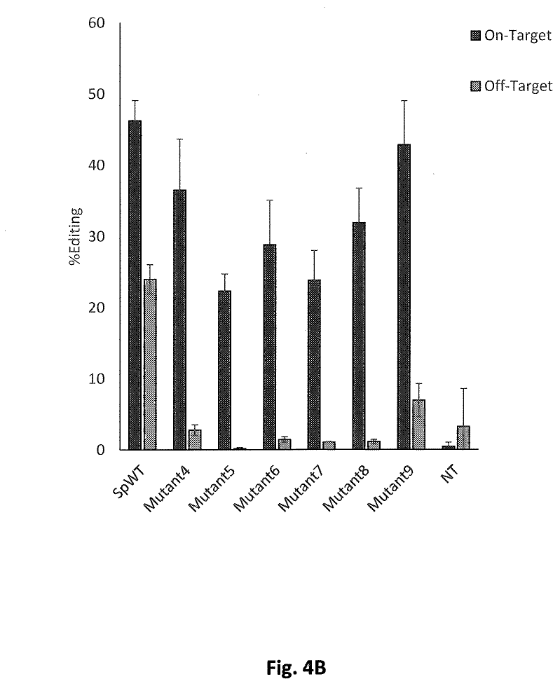

In all tested sites, WT-SpCas9 editing is observed on the expected target site, however significant editing can also be observed at other non-related genomic location (i.e. off-target sites), therefore its specificity is lower than that of the tested variants (for example, see FIG. 2B, FIG. 3, and FIG. 4).

TABLE 5
Summary of amino acid positions 929 and 930 for SpCas9 and
variants of SpCas9 (including V10, which is Variant 1).

| Variant Name | Position 929 | Position 930 |
|---|---|---|
| SpCas9 WT | K | H |
| Variant 1 (V10) | T | A |
| Variant 2 | K | A |
| Variant 3 | T | H |
| Variant 4 | T | Y |
| Variant 5 | T | D |
| Variant 6 | T | T |
| Variant 7 | Y | A |
| Variant 8 | D | A |
| Variant 9 | A | A |

TABLE 6
Sites tested for editing

| | | On target | Off target |
|---|---|---|---|
| | Spacer | amplicon | amplicon |
| CXCR4 | SEQ ID NO: 40 | SEQ ID NO: 41 | SEQ ID NO: 42 |
| ELANE g35 | SEQ ID NO: 43 | SEQ ID NO: 44 | SEQ ID NO: 45 |
| ELANE g58_alt | SEQ ID NO: 46 | SEQ ID NO: 47 | SEQ ID NO: 48 |
| EMX | SEQ ID NO: 49 | SEQ ID NO: 50 | SEQ ID NO: 51 |

Sequences of Variants 1-10 are shown below, with positions 929 and 930 underlined:

Variant 1 (V10) SpCas9 amino acid sequence
(SEQ ID NO: 22)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITTAVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
Variant 2 SpCas9 amino acid sequence
(SEQ ID NO: 23)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKAVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
Variant 3 SpCas9 amino acid sequence
(SEQ ID NO: 24)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITTHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
Variant 4 SpCas9 amino acid sequence
(SEQ ID NO: 25)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITTYVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
Variant 5 SpCas9 amino acid sequence
(SEQ ID NO: 26)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITTDVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
Variant 6 SpCas9 amino acid sequence
(SEQ ID NO: 27)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITTTVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
Variant 7 SpCas9 amino acid sequence
(SEQ ID NO: 28)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITYAVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
Variant 8 SpCas9 amino acid sequence
(SEQ ID NO: 29)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITDAVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
Variant 9 SpCas9 amino acid sequence
(SEQ ID NO: 30)
MDKKYSIGLDIGTNSVGWAVITDEYKVPSKKEKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECEDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITAAVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

1. A non-naturally occurring SpCas9 variant having an amino acid substitution at position K929, position H930, or at both positions K929 and H930.

2. The SpCas9 variant of claim 1, wherein the amino acid substitution at position K929 is selected from the group consisting of R, H, D, E, S, T, N, Q, C, U, G, P, A, I, L, M, F, W, Y, and V,
wherein the amino acid substitution at position K929 is an uncharged, negative, polar or non-polar amino acid, or
wherein the amino acid substitution at position K929 is selected from the group consisting of K929T, K929Y, K929D, and K929A.

3-4. (canceled)

5. The SpCas9 variant of claim 1, wherein the amino acid substitution at position H930 is selected from the group consisting of R, D, E, S, T, N, Q, C, U, G, P, A, I, L, M, F, W, Y, and V,
wherein the amino acid substitution at position K930 is an uncharged, negative, polar or non-polar amino acid, or
wherein the amino acid substitution at position H930 is selected from the group consisting of H930A, H930Y, H930D, and H930T.

6-7. (canceled)

8. The SpCas9 variant of any one of claim 1, having an amino acid sequence selected from the group consisting of SEQ ID NOs: 22-30.

9. The SpCas9 variant of claim 1, having at least 80% sequence identity to the wild-type SpCas9 amino acid sequence listed as SEQ ID NO: 1.

10. The SpCas9 variant of claim 1, further comprising a nuclear localization sequence (NLS).

11. The variant SpCas9 of claim 1, wherein the variant exhibits increased specificity toward a DNA target site when complexed with a gRNA that targets the said DNA target site compared to a wild-type SpCas9 complexed with the gRNA.

12. A CRISPR/Cas system comprising the variant SpCas9 of claim 1 complexed with a gRNA that targets a DNA target site, wherein the CRISPR/Cas system displays reduced off-target editing activity relative to a wild-type CRISPR/Cas system comprising a wild-type SpCas9 protein and the gRNA.

13. A method for gene editing having reduced off-target editing activity, comprising contacting a DNA target site with an active CRISPR/Cas system comprising a variant SpCas9 of claim 1, wherein the active CRISPR/Cas system displays reduced off-target editing activity relative to a wild-type CRISPR/Cas system comprising a wild-type SpCas9 protein.

14. The method of claim 9, wherein the gene editing occurs in a eukaryotic cell.

15. The method of claim 9, wherein in the cell is a plant cell or mammalian cell.

16. The method of claim 9, wherein the DNA target site is located within or in proximity to a pathogenic allele of a gene.

17. The method of claim 9, wherein the DNA target site is located in a gene selected from the group consisting of ELANE, CXCR4, EMX, RyR2, KNCQ1, KCNH2, SCN5a, GBA1, GBA2, Rhodopsin, GUCY2D, IMPDH1, FGA, BEST1, PRPH2, KRT5, KRT14, ApoA1, STAT3, STAT1, ADA2, RPS19, SBDS, GATA2, and RPE65.

18. The method claim 9, wherein the DNA target is repaired with an exogenous donor template.

19. The method of claim 9, wherein the off-target editing activity is reduced by at least 2-fold, 10-fold, 10²-fold, 10³-fold, 10⁴-fold, 10⁵-fold, or 10⁶-fold.

20. A modified cell obtained by the method of claim 9.

21. The modified cell of claim 16, wherein the cell is capable of engraftment,
wherein the cell is capable of giving rise to progeny cells after engraftment,
wherein the cell is capable of giving rise to progeny cells after an autologous engraftment, and/or
wherein the cell is capable of giving rise to progeny cells for at least 12 months or at least 24 months after engraftment.

22-24. (canceled)

25. The modified cell of claim 16, wherein the cell is selected from the group consisting of a hematopoietic stem cell, a progenitor cell, a CD34+ hematopoietic stem cell, a bone marrow cell, and a peripheral mononucleated cell.

26. A composition comprising a modified cell of claim 16 and a pharmaceutically acceptable carrier.

27. An in vitro or ex vivo method of preparing the composition of claim 19, comprising mixing the cells with the pharmaceutically acceptable carrier.
